(19) United States
(12) Patent Application Publication
(10) Pub. No.: US 2002/0068039 A1
(43) Pub. Date: Jun. 6, 2002

(54) Oral care compositions containing grapefruit seed extract

(76) Inventors: Pauline C. Pan, Denville, NJ (US); Mary Beth Finnegan, Hillsborough, NJ (US)

Correspondence Address: PFIZER, INC., 201 TABOR ROAD, MORRIS PLAINS, NJ, 07950, US

(21) Appl. No.: 09/881,373

(22) Filed: Jun. 14, 2001

Related U.S. Application Data
(60) Provisional application No. 60/221,312, filed on Jul. 28, 2000.

(51) Int. Cl. A61K 7/18; A61K 7/26

(52) U.S. Cl. 424/52; 424/58

(57) ABSTRACT

Oral care compositions, such as mouthwashes and toothpastes, include grapefruit seed extract in synergistic combination with an ion-providing compound. The combined ingredients are synergistically effective to inhibit growth and metabolism of and kill plaque bacteria. The ion-providing compound can provide fluoride, a cationic antimicrobial agent and/or a cationic surfactant.

This application claims the benefit of U.S. Provisional Patent Application No. 60/221312, filed on Jul. 28, 2000, the entirety of which is hereby incorporated by reference as if fully set forth herein.

### FIELD OF THE INVENTION

This invention relates to oral care compositions containing citrus fruit extracts and more particularly to oral care compositions containing grapefruit seed extract.

### BACKGROUND OF THE INVENTION

Consumer demand for so-called “all-natural” products is strong and shows no signs of waning. The appeal of all-natural products is broad enough to encompass largely artificial products containing at least some natural ingredients, particularly where the natural ingredient is purported to provide the essential activity to the product. One such “all-natural” active ingredient being employed in a growing variety of contexts is grapefruit seed extract (GSE).

For example, U.S. Pat. Nos. 3,852,436, 3,890,212, 4,021,548, 4,021,577, 4,021,578, 5,425,944, and 5,631,001 to Harich or Harich et al., which patents are herein incorporated by reference, disclose various compositions comprising GSE. GSE is purported to have antimicrobial activity comparable to that of more traditional, synthetic antimicrobial agents. Although the '944 and '001 patents disclose oral care compositions (i.e., mouthwashes) comprising 50-100 ppm of GSE, none of the Harich patents disclose oral care compositions comprising fluoride and GSE.

U.S. Pat. No. 5,378,465 to Zeines discloses a mouthwash comprising an aqueous solution containing 0.20% GSE. The mouthwash contains significant amounts of alcohol. Zeines does not disclose the use of fluoride in combination with GSE in an oral care composition.

U.S. Pat. No. 5,128,139 to Brown et al. discloses compositions comprising liposomes containing GSE and triclosan. The compositions are particularly suitable for use as deodorants.

U.S. Pat. No. 4,420,471 to Elton et al. discloses mouthwashes comprising about 0.01 to 0.5 wt. % citrus oil, such as grapefruit oil. This patent teaches that alcohol is necessary to help solubilize the citrus oil. Elton et al. does not disclose oral care compositions comprising GSE.

JP Patent Application No. 97-143084 discloses anti-cariogenic foods comprising extracts of citrus fruits other than grapefruit. Oral care compositions comprising GSE are not disclosed.

Although the prior art contemplates oral care products containing GSE and oral care products containing fluoride, it does not appear that the art teaches combining synergistically effective amounts of these ingredients to form a oral care product synergistically effective to reduce growth of, inhibit metabolize of and kill germs that cause tooth decay and plaque formation.

It is therefore desired to provide an oral care composition that is anticaries, wherein the composition contains GSE and fluoride in a combination synergistically effective to reduce growth of, inhibit metabolize of and kill germs that cause tooth decay and plaque formation.

All references cited herein are incorporated herein by reference in their entireties.

### SUMMARY OF THE INVENTION

The invention provides an oral care composition comprising:

a grapefruit seed extract;

an ion-providing compound selected from the group consisting of fluorine-providing compounds and cation-providing compounds;

a flavorant; and

a pharmaceutically acceptable vehicle,

wherein said grapefruit seed extract and said ion-providing compound are synergistically effective to reduce growth of, inhibit metabolism of and kill plaque bacteria.

Also provided is a method for treating an oral cavity to reduce plaque, said method comprising applying to a surface of said oral cavity an oral care composition according to the invention.

### BRIEF DESCRIPTION OF THE DRAWINGS

The invention will be described in conjunction with the following drawings in which like reference numerals designate like elements and wherein:

FIG. 1A is a graph of pH versus duration of exposure to several oral care compositions;

FIG. 1B is a graph of lactic acid concentration versus duration of exposure to several oral care compositions;

FIG. 2A is a graph of pH versus duration of exposure to several oral care compositions; and

FIG. 2B is a graph of lactic acid concentration versus duration of exposure to several oral care compositions.

### DETAILED DESCRIPTION OF PREFERRED EMBODIMENTS

The invention provides novel oral care compositions comprising GSE in amounts ranging from about 0.001% to about 10%, preferably from about 0.01% to about 1%, more preferably from about 0.2% to about 1% (wherein the percentages are weight/volume values based on the percent actives in commercially available GSE).

The GSE of the invention is preferably provided in accordance with the teachings of U.S. Pat. Nos. 3,852,436, 3,890,212, 4,021,548, 4,021,577, 4,021,578, 5,425,944, and 5,631,001.

Accordingly, the starting material of the GSE preparation process comprises grapefruit pulp and/or grapefruit seeds. Pulp is located immediately under the hard, outer rind layer of the skin of fresh grapefruit and is obtained by mechanically shaving the rind portion from the skin, normally after the inner juice, meat and section skins have been previously removed. The separation of the rind from the inner pulp layer of the skin should be accomplished in such a manner that the inner pulps are not damaged.

The pulps used are preferably acquired from fresh, ripe grapefruit obtained when the acid content of the fruit is low, as shown by a pulp pH of about 2.5 to 5.0, and preferably 3.5 to 5.0. The pulp is preferably obtained during the December through April grapefruit season in Florida, and used while it is fresh, for example, after storage at 40 to 45° F. (4 to 7° C.) for a period of not over about three (3) days. Longer storage times, up to several months or longer, can be achieved by adding the alcohol or ketone reactant, e.g., propylene glycol or glycerin, to the pulp and storing the two together. The grapefruit pulp is reacted with the alcohol or ketone, preferably at an elevated temperature and under the influence of ultraviolet radiation, to produce a stable reaction product. Both monohydric and polyhydric alcohols can be used in this first stage reaction. Thus, suitable alcohols include methanol, ethanol, isopropanol, n-propanol, n-butanol, allyl alcohol, amyl alcohol, tert-amyl alcohol, octyl alcohol, benzyl alcohol, ethylene glycol, propylene glycol, diethylene glycol, dipropylene glycol, triethylene glycol, tetraethylene glycol, glycerin and the like. Acetone is the presently preferred ketone reactant.

The polyhydric, aliphatic alcohols, such as propylene glycol and glycerin, are greatly preferred reactants in the present process, and, in fact, appear to yield significantly superior results over the other alcohol or ketone reactants which can also be used.

The reaction is carried out at room temperature or above, with the reaction proceeding more rapidly at somewhat elevated temperatures. Reaction temperatures of 90 to 140° F. (32 to 60° C.) are generally used, and temperatures of 110 to 120° F. (43 to 49° C.) are preferred.

The ratio of alcohol or ketone to grapefruit pulp used in the reaction can be varied widely. Most of the alcohol used in the preferred procedure described below does not enter into a reaction with the pulp and is believed to serve only a mechanical or extractive function, if any. It is to be understood that the term “reaction” as used here is intended to have its broadest meaning and includes extractive reactions or any other chemical mechanism that may occur as a result of the practice of the first step of the present process. An excess of alcohol (i.e., propylene glycol) reactant is presently preferred and the reaction is generally carried out using a weight ratio of grapefruit pulp to propylene glycol or glycerin of about 1:2. This ratio of reactants has been generally found to yield a superior quality and quantity of intermediate reaction product. Depending on many factors such as frosts, application of pesticide to the fruit, etc., it may be desirable in some cases to vary the ratio of pulp to alcohol as low as 1:4.5 or even lower.

The reaction is preferably carried out in the presence of a free radical initiator, most preferably ultraviolet (UV) light. Other conventional radical initiators, such as the chemical initiators tertiary butyl hydroperoxide, azobisbutyronitrile, dicumyl peroxide or the like, can also be used. UV light has been found to function well in the present process and is presently preferred. The UV light may be supplied by commercially available UV light sources or even by sunlight.

In embodiments, GSE is provided in accordance with U.S. Pat. No. 5,425,944. Accordingly, the GSE of these embodiments is provided by separating the seed and pulp of grapefruit from the remainder of the fruit and drying the seed and pulp for 24-48 hours at a temperature in the range of 150-200° C. The seed and pulp are then tested for pesticides and only non-contaminated seed and pulp are selected for processing. The dried, non-contaminated seed and pulp is mixed at a ratio of 80:20 seed/pulp by weight. The mixture is then ground in a hammermill to small particles, whereupon the ground mixture is placed inside some type of mesh bag or perforated container which is then placed in a reaction vessel. Previously, a glycerin solution in an amount by weight approximately equal to the weight of the ground mixture has been placed in the vessel and heated to a temperature of at least approximately 150° C. The vessel is sealed and the glycerin solution is then circulated through the extraction chamber and past an external ultraviolet system and magnetic system which helps to stabilize the ingredients and to remove ferrous metallic particles therefrom. The glycerin circulation is continued at the same temperature for approximately 3-4 hours, whereupon the temperature is reduced to approximately 60° C. while the pressure within the chamber is increased to a range of 2,500-3,000 lbs/sq. inch. There results a syrup and a residue in the reaction chamber. The syrup is then passed through a force filter system having a 300-350 mesh nylon filter to obtain a heavy viscous lemon yellow liquid having a pH in the range of 2.5-3.0. This is the reaction product that is either then diluted and used in various applications or else dehydrated and used in various applications.

Compositions of the invention include ingredients additional to GSE, such as, e.g., additional antimicrobial agents, fluorine-providing compounds, acidifiers, abrasives, surfactants, binders, thickeners, humectants, sweeteners, desensitizing agents, flavorants, colorants, and preservatives. The ingredients are combined in a hydrous or anhydrous vehicle to form a solid (e.g., a toothpowder), a semi-solid (e.g., a paste or a gel), a liquid (e.g., a mouthwash), a rapidly dissolving orally consumable film, a chewable tablet, a capsule, a foam and other known oral composition forms.

In certain compositions according to the invention, a fluorine-providing compound and GSE are combined to synergistic effect. The synergistic effect relates to inhibiting and reducing the growth of microbes and inhibiting the metabolism of and killing bacteria, e.g., plaque bacteria, which is achieved when the fluorine-providing compound and GSE are utilized in combination in effective concentrations in the oral cavity. Smaller quantities of each of these components are required to obtain effective inhibition of plaque bacteria and other microbes than if each component were utilized alone. Inhibition of bacteria means reducing the growth of, inhibiting the metabolism of and killing the bacteria. Since lower quantities of each component can be used in the compositions of this invention, the side effects associated with each of the components may correspondingly be reduced or eliminated.

Synergistically effective compositions of the invention comprise fluorine-providing compounds in amounts ranging from about 0.001 wt. % to about 1.0 wt. %, preferably from about 0.05 wt. % to about 0.075 wt. %, more preferably from about 0.015 wt. % to about 0.05 wt. %. The ratio (w/w) of the active components of GSE to fluorine-providing compounds is about 10:2, preferably about 8:2, more preferably about 6:1.

The fluorine-providing compounds can be slightly water soluble or fully water soluble and are characterized by their ability to release fluoride ions or fluoride containing ions in water. Suitable fluorine-providing compounds include, e.g., inorganic fluoride salts, such as soluble alkali metal, alkaline earth metal, and heavy metal salts, e.g., sodium fluoride, potassium fluoride, ammonium fluoride, cuprous fluoride, zinc fluoride, stannic fluoride, stannous fluoride, barium fluoride, sodium fluorosilicate, ammonium fluorosilicate, sodium fluorozirconate, sodium monofluorophosphate, aluminium mono-and difluorophosphate and fluorinated sodium calcium pyrophosphate.

In certain compositions according to the invention, a cation-providing compound and GSE are combined to synergistic effect. The synergistic effect relates to inhibiting and reducing the metabolism and growth of microbes, such as plaque bacteria, which is achieved when the cation-providing compound and GSE are utilized in combination in effective concentrations in the oral cavity. Smaller quantities of each of these components are required to obtain effective inhibition of plaque bacteria and other microbes than if each component were utilized alone. Since lower quantities of each component can be used in the compositions of this invention, the side effects associated with each of the components are correspondingly reduced or eliminated.

Synergistically effective compositions of the invention comprise cation-providing compounds in amounts ranging from about 0.001 wt. % to about 1.0 wt. %, preferably from about 0.005 wt. % to about 0.3 wt. %, more preferably from about 0.01 wt. % to about 0.10 wt. %. The ratio (w/w) of GSE to cation-providing compounds is about 10:1, and preferably about 3:1.

The cation-providing compounds are preferably cationic antimicrobial agents and/or cationic surfactants, such as, e.g., cetylpyridinium chloride, domiphen bromide, chlorhexidene, chitosan, ethyl N^{α}-lauryl-L-arginate pyrrolidone-5-carboxylic acid salt (hereinafter LAE) and quaternary ammonium salts.

In embodiments, the oral composition may be a liquid such as a mouthwash or rinse. The total amount of the liquid vehicle in a mouthwash composition is typically in the range of about 70% to about 99.9% by weight of the composition. The pH value of such mouthwash compositions is generally from about 4.0 to about 8.5 and preferably from about 4 to about 7.5. A pH below 4 would be irritating to the oral cavity. A pH greater than 8.5 would result in an unpleasant mouth feel.

In embodiments, the vehicle is a water-alcohol mixture, wherein the ratio of water to alcohol is in the range of from about 1:1 to about 20:1, preferably about 3:1 to about 20:1 and most preferably about 3:1 to about 10:1 by weight. The most preferred mouthwash or mouth rinse compositions comprise from 0 to about 30% by weight alcohol, such as ethanol.

In certain preferred embodiments, the oral care composition is alcohol-free. In these embodiments, alcohol is not used as the vehicle for the composition. The expression “alcohol-free” is not intended to exclude any minor amounts of alcohol contributed by GSE (e.g., as an artifact of its purification) or flavor oils. Although the prior art teaches that alcohol is necessary to adequately deliver antimicrobials to plaque, the inventors have overcome this limitation in providing alcohol-free compositions comprising 0.001 to 2% GSE. It is surprising that an oral care composition effective against bacterial plaque can be provided without alcohol in view of the understanding in the prior art that alcohol is necessary as a vehicle to ensure penetration of the hydrophilic matrix of the plaque and the lipophilic membrane of the plaque bacteria.

Oral liquid compositions can also contain surface active agents in amounts up to about 5%. Surface active agents are organic materials which afford complete dispersion of the composition throughout the oral cavity. The organic surface active material can be non-ionic, amphoteric, or cationic (with cationic being preferred).

Non-ionic surface active agents include condensates of sorbitan mono-oleate with from 20 to 60 moles of ethylene oxide (e.g., “Tweens” a trademark of ICI United States, Inc.), condensates of ethylene oxide with propylene oxide and condensates of propylene glycol (“Pluronics” a trademark of BASF-Wyandotte Corp.).

Other suitable non-ionic surfactants useful in the present invention include polyoxyethylene castor oil derivatives which are ethoxylated hydrogenated castor oils. These surfactants are prepared by hydrogenating castor oil and treating the hydrogenated product with from about 10 to about 200 moles of ethylene glycol. These ethoxylated hydrogenated castor oils are known by the non-proprietary name of polyethylene glycol (PEG) hydrogenated castor oils, in accordance with the Dictionary of the Cosmetics, Toiletries and Fragrance Association, 3rd Edition, which name is used in conjunction with a numeric suffix to designate the degree of ethoxylation of the hydrogenated castor oil product, i.e., the number of moles of ethylene oxide added to the hydrogenated castor oil product. Suitable PEG hydrogenated castor oils include PEG 16, 20, 25, 30, 40, 50, 60, 80, 100 and 200. A preferred PEG hydrogenated castor oil surfactant is Cremophor RH 60, a commercially available product from BASF-Wyandotte, Parsippany, N.J.

Other suitable non-ionic surfactants are the condensation products of an alpha-olefin oxide containing 10 to 20 carbon atoms, a polyhydric alcohol containing 2 to 10 carbons and 2 to 6 hydroxyl groups and either ethylene oxide or a mixture of ethylene oxide and propylene oxide. The resultant surfactants are heteric polymers having a molecular weight in the range of about 400 to about 1600 and containing 40% to 80% by weight of ethylene oxide, with a alpha-olefin oxide to polyhydric alcohol mole ratio in the range of about 1:1 to 1:3.

Amphoteric surfactants useful in the present invention include zwitterions having the capacity to act as either an acid or a base. They are generally non-irritating and non-staining. Non-limiting examples of suitable amphoteric surfactants include cocoamidopropyldimethylsultaine and cocodimethylbetaine (commercially available from Lonza Chem. Co. under the trade-names Lonzaine CS and Lonzaine 12C, respectively).

Cationic surface active agents suitable for use in the invention include, e.g., quaternary ammonium compounds. As mentioned above, cationic ingredients, such as cationic surfactants, can synergistically enhance the antimicrobial activity of the oral care composition of the invention.

The compositions of this invention may be substantially solid or pasty in character such as dental cream, toothpaste, toothpowder or chewing gum. Solid or pasty oral compositions contain polishing materials. Typical polishing materials are abrasive particulate materials having particle sizes of up to about 20 microns. Non-limiting illustrative examples include water-insoluble sodium metaphosphate, potassium metaphosphate, tricalcium phosphate, dihydrated calcium phosphate, anhydrous dicalcium phosphate, dicalcium phosphate, calcium pyrophosphate, magnesium orthophosphate, trimagnesium phosphate, calcium carbonate, alumina, aluminum silicate, zirconium silicates, silica, bentonite, and mixtures thereof. Polishing materials are generally present in an amount from about 20% to about 99% by weight of the composition. Preferably, it is present in amounts from about 20% to about 75% in toothpaste, and from about 70% to about 99% in toothpowder.

In clear gels, it is preferred to provide a polishing agent of colloidal silica and alkali metal aluminosilicate complexes since they have refractive indices close to the refractive indices of gelling agent liquid systems commonly used in dentifrices.

The compositions of the present invention may additionally contain sweeteners, flavorants, colorants and other known oral care ingredients such as other anti-microbial agents, anti-tartar agents, desensitizing agents, saliva stimulating agents and the like.

In the instance where auxiliary sweeteners are utilized, the present invention contemplates the inclusion of those sweeteners well known in the art, including both natural and artificial sweeteners. Thus, additional sweeteners may be chosen from the following non-limiting list:

A. Water-soluble sweetening agents, such as monosaccharides, disaccharides and polysaccharides, such as xylose, ribose, glucose, mannose, galactose, fructose, dextrose, sucrose, maltose, partially hydrolyzed starch, or corn syrup solids and sugar alcohols such as sorbitol, xylitol, mannitol and mixtures thereof.

B. Water-soluble artificial sweeteners, such as the soluble saccharin salts, i.e., sodium, or calcium saccharin salts, cyclamate salts, acesulfame-K and the like, and the free acid form of saccharin.

C. Dipeptide based sweeteners such as L-phenylalanine methyl ester and materials described in U.S. Pat. No. 3,492,131 and the like.

In general, the amount of sweetener will vary with the desired amount of sweetness selected for a particular composition. This amount will normally be 0.01% to about 40% by weight. The water-soluble sweeteners described in category A above, are preferably used in amounts of about 5% to about 40% by weight, and most preferably from about 10% to about 20% by weight of the final composition. In contrast, the artificial sweeteners described in categories B and C are preferably used in amounts of about 0.005% to about 5.0% and most preferably about 0.05% to about 2.5% by weight of the final composition. These amounts are ordinarily necessary to achieve a desired level of sweetness independent from the flavor level achieved from flavorants.

Suitable flavorants include, e.g., both natural and artificial flavors, such as mints (e.g., peppermint spearmint, etc.), citrus flavors such as orange and lemon, artificial vanilla, cinnamon, various fruit flavors and the like. Both individual and mixed flavors are contemplated. The flavorings are generally utilized in amounts that will vary depending upon the individual flavor, and can, for example, range in amounts of about 0.1% to about 6% by weight of the final composition.

The colorants useful in the present invention include pigments which can be incorporated in amounts of up to about 2% by weight of the composition. Also, the colorants can include other dyes suitable for food, drug and cosmetic applications (i.e., FD&C dyes) and the like. The materials acceptable for the foregoing spectrum of use are preferably water-soluble. Illustrative examples include the indigo dye known as FD&C Blue No. 2, which is the disodium salt of 5,5-indigotindisulfonic acid, FD&C Green No. 1, which is a triphenylmethane dye and is the monosodium salt of 4-[4-N-ethyl-p-sulfobenzyl amino)diphenyl-methylene]-[1-(N-ethyl-N-p-sulfoniumbenzyl)-2,5-cyclohexadie nimine]. A full recitation of all FD&C and D&C colorants useful in the present invention and their corresponding chemical structures can be found in the Kirk-Othmer Encyclopedia of Chemical Technology, 3rd Edition, in Volume 6, at pages 561-595.

The present invention also involves a method for treating teeth or gums to reduce plaque, comprising applying to the surface of the teeth and/or gums the compositions of this invention as described earlier. The compositions can be applied to the teeth and gums by any conventional means, such as brushing, spraying, painting or rinsing of the oral cavity and the like. The invention will be illustrated in more detail with reference to the following Examples, but it should be understood that the present invention is not deemed to be limited thereto.

### EXAMPLE 1

### Base Formulation

A base was formulated for Grapefruit Seed Extract (GSE). It was discovered that GSE is not compatible with saccharin or acesulfame K directly. It is compatible with aspartame, but the combination is commercially impractical due to a relatively short shelf life. A solution of GSE, saccharin and 0.01% CPC is clear. A mouthwash base was formulated without flavor oils and antimicrobial activity was tested.

TABLE 1

| | % w/v | wt/L |
|---|---|---|
| Citric Acid | 0.0035 | 0.035 |
| Na citrate | 0.25 | 2.500 |
| Sodium Fluoride | 0.05 | 0.5 |
| Poloxamer 407 | 0.6 | 6.0 |
| Sorbitol (70%) | 12% (final) | 171.4 |
| GSE (active component) | | |
| 52.3% Active | 0.35% | 6.7 |
| Glycerin (from GSE) | 0.268% | |
| CPC | 0.01 | 0.1 |
| Na saccharin | 0.04 | 0.4 |

The resulting composition had a pH of 6.57 and a slightly yellow color.

### Plaque Penetration Assay

The plaque penetration assay employed by the present inventors was a modification of the well-known procedure described or referenced in, e.g., Tanzer et al., “In Vitro Evaluation of Seven Cationic Detergents as Antiplaque Agents,” Antimicrobial Agents and Chemotherapy, March 1979, pp. 408-414.

The ethanol mouthwash composition employed by the present inventors as their standard for their plaque penetration assay contained 27% v/v ethanol and had the composition shown in Table 2:

TABLE 2

| Ingredient | Amount |
|---|---|
| Ethanol (USP) | 284 mls |
| Thymol | 0.64 gram |
| Eucalyptol | 0.92 gram |
| Menthol | 0.42 gram |
| Methyl Salicylate | 0.60 gram |
| Benzoic Acid | 1.5 grams |
| Caramel | 0.2 gram |
| Poloxamer 407 | 1.0 |
| Water | Q.S. to 1 Liter |

The media required for the plaque penetration assay included sterile deionized water, Letheen Broth (DIFCO) and modified Jordan's complex medium (with and without bromocresol purple pH indicator) (see Jordan et al., J. Dent. Res. 39: 116-123 (1960)).

Jordan's medium was prepared by blending the ingredients listed in Tables 3 and 4:

TABLE 3

| Ingredient | Amount |
|---|---|
| Trypticase Peptone (BBL) | 5 grams |
| Yeast Extract | 5 grams |
| K₂HPO₄ | 5 grams |
| Stock Salts Solution (see below) | 0.5 ml |
| Sucrose | 50 grams |
| Sodium Carbonate | 0.05 grams |
| Deionized Water | Q.S to 1 Liter |

TABLE 4
Stock Salts Solution

| Ingredient | Concentration |
|---|---|
| MgSO₄ (anhydrous) | 3.9 g/L |
| FeCL₃.6H₂O | 0.4 g/L |
| MnCl₂ (anhydrous) | 0.12 g/L |
| Distilled water | Q.S. to 1 Liter |

The pH was adjusted to 7.2 with 5N HCl. Jordan's medium with pH indicator (“recovery medium”) was prepared by adding 1 ml of a 1% bromocresol purple stock solution (i.e., 0.1 gram bromocresol purple in 10 mls distilled water) to 1 liter of Jordan's medium.

### Biofilm Formation

The culture for the assay was prepared as follows. S. mutans UA159 was inoculated in the previously described Jordan's complex medium and incubated for 20 hours at 33° C. After 20 hrs, media above biofilm were removed and replaced with 250 microliters of fresh media. All 96 well microliter plates were then incubated an additional 4 hours. The in situ formed biofilm was then used for assessing activity.

### Assay Procedure

Each sample was run in quadruplicate for each time point. All solutions and plates were kept at 33° C. The control standard was run with each formulation tested. Media were removed and replaced with 250 microliters of sterile water and incubated for 2 minutes. Water was removed and test mouthrinses were added to each well. The biofilms were exposed to mouthwash for 1, 2, 3 and 4 minutes. After the appropriate incubation time, the test rinse was removed from above the biofilm and replaced with Letheen Broth. Letheen Broth served as the stop solution to inactivate the rinses. Letheen Broth was removed after it incubated in the wells for at least 5 minutes. Then, 250 microliters of indicator medium were added to all wells. After the entire plate was run, it was then incubated at 33° C. for 18 hours. After the 18-hour post assay incubation, results were analyzed calorimetrically. If the organisms in the film were viable, they produced lactic acid, which caused the indicator in the growth medium to change from purple to yellow. The continued presence of purple in the well indicated that no growth of bacteria had occurred. The results can also be determined by measuring the pH of the final medium. If the bacteria produced lactic acid, the pH of the medium should be reduced. In addition, the lactic acid in the medium can be measured directly, using Sigma Diagnostic Lactate Kit No. 735.

### Microtiter Method Using Sigma Diagnostics Lactate Kit No.735

Lactic acid is converted to pyruvate and hydrogen peroxide by oxidase. In the presence of hydrogen peroxide formed, peroxidase catalyzes the oxidative condensation of chromogen precursors to produce a colored dye with an absorption maximum at 540 nm. The increase in absorbance at 540 nm is directly proportional to lactate concentration in the sample.

A standard curve was generated according to the following steps:

1. Pipette out 5 μl of the three given standards, 20, 80 and 120 mg/dL in triplicates in 96 well microtiter plates.

2. Dilute the 20 mg/dL standard 1:1 to give a 10 mg/dL standard.

3. To the standards, 250 μl of the lactate reagent were added. The lactate reagent was prepared by dissolving the reagent in 10 ml of DI water.

4. The plate was incubated at room temperature for 10-15 minutes.

5. The absorbance was measured at 540 nm in a spectrophotometer. The plate can be read up to an hour after the lactate reagent is added.

Samples being evaluated were tested according to the following steps:

1. Pipette out 5 μl of each sample onto a 96 well microtiter plate.

2. To the 5 μl of sample, 250 μl of the lactate reagent was added and incubated at room temperature for 10 to 15 minutes.

3. After the incubation, the plate is read at 540 nm.

The increase in the absorbance at 540 nm is directly proportional to the lactate concentration in the sample.

### Colorimetric Determination of Critical Kill Times and R-Factor

The critical time necessary for the sample to completely kill the microorganism can be determined by observing the point (front to back or bottom to top, as the case may be) at which the Jordan's recovery medium color changed from yellow to purple. The critical kill time for any sample, divided by the critical kill time for the control mouthwash in that same rack, gives the R-Factor for that sample.

Table 5 summarizes a statistical scale which relates the observed change from growth (+) to no growth (−) to critical kill times. For example, as shown in the first row of the table, where the observed condition changes from growth (continuous +'s) to no growth (continuous −'s) (“no anomaly”), the critical kill time is determined by adding 0.50 minute to the time at which the last growth observation (+) was made. The balance of Table 2 sets forth how critical kill times are determined for different observed growth/no growth intervals between continuous growth segments and continuous no growth segments.

TABLE 5
BUSCH Scores for Critical Kill Times (CKT)

| Intervals between continuous +'s and −'s | Add To Last (+) Time |
|---|---|
| No anomaly | 0.50 |
| −+ | 1.50 |
| −++ | 2.90 |
| −+++ | 4.10 |
| −+−+ | 2.50 |
| −+−−+ | 2.10 |
| −++−+ | 4.06 |
| −−+ | 1.10 |
| −−++ | 2.50 |
| −−+−+ | 3.84 |
| −−−+ | 0.90 |
| −−−−+ | 0.80 |

By way of further example, consider the examples of growth/no growth sequences, and their associated critical kill times, in Table 6. In the first row of Table 6, there was no fanomaly between continuous +'s and continuous −'s; therefore, CKT (per Table 5)=4.0+0.5=4.5 minutes (i.e., kill occurred somewhere between 4.0 and 5.0 minutes). In the second row of Table 6, the interval between continuous +'s and continuous −'s is −+; therefore, CKT (per Table 5)=2.0+1.5=3.5 minutes.

TABLE 6
Examples of Growth/No Growth Sequences and CKT

Treatment Times (min)

| | 2 | 3 | 4 | CKT |
|---|---|---|---|---|
| + | + | + | − | 3.5 |
| + | − | + | − | 2.5 |
| + | + | + | + | >4.5 |
| − | − | − | − | <0.5 |
| + | − | − | + | 2.1 |

In the case of rows 3 and 4 of Table 6, clearly no end point was reached. It is assumed here that kill will occur at some point in excess of 6.5 minutes (>6.5) or much below 2.0 minutes (<2.0), respectively.

Row 5 of Table 6 is an example where the kill scale is dependent on the values which are located to the left of the last + and to the right of the first −. For that particular example, CKT=2.0+1.1=3.1 minutes (per Table 5).

The following compositions were tested in accordance with the foregoing techniques:

(a) 0.3% Grapefruit Seed Extract, pH 4.2

Based on the certificate of analysis of the batch used, 52.3% of total weight was the active component. Therefore, for 0.3% active component, 0.57 g of liquid extract was diluted to a 100 ml total volume. The pH of the solution was adjusted to 4.2 with 1N NaOH.

(b) 0.3% Grapefruit Seed Extract/0.05% Na Fluoride, pH 4.2

Based on the certificate of analysis of the batch used, 52.3% of total weight was the active component. Therefore, for 0.3% active component, 0.57 g of liquid extract and 0.05 g of sodium fluoride were diluted to 100 ml total volume. The pH of the solution was adjusted to 4.2 with 1N NaOH.

(c) 0.05% Sodium Fluoride, pH 4.2

Sodium fluoride (0.05 g) was dissolved to 100 ml total volume. The pH of the solution was adjusted to 4.2 with 1N NaOH.

(d) 0.3% Grapefruit Seed Extract, pH 6.0

Based on the certificate of analysis of the batch used, 52.3% of total weight was the active component. Therefore, for 0.3% active component, 0.57 g of liquid extract was diluted to 100 ml total volume. The pH of the solution was adjusted to 6.0 with 1N NaOH.

(e) 0.3% Grapefruit Seed Extract/0.05% Na Fluoride, pH 6.0

Based on the certificate of analysis of the batch used, 52.3% of total weight was the active component. Therefore, for 0.3% active component, 0.57 g of liquid extract and 0.05 g of sodium fluoride were diluted to a 100 ml total volume. The pH of the solution was adjusted to 6.0 with 1N NaOH.

(f) 0.05% Sodium Fluoride, pH 6.0

Sodium fluoride (0.05 g) was dissolved to 100 ml total volume. The pH of the solution was adjusted to 6.0 with 1N NaOH.

(g) Deionized, sterile water.

(h) ethanol formulation described above.

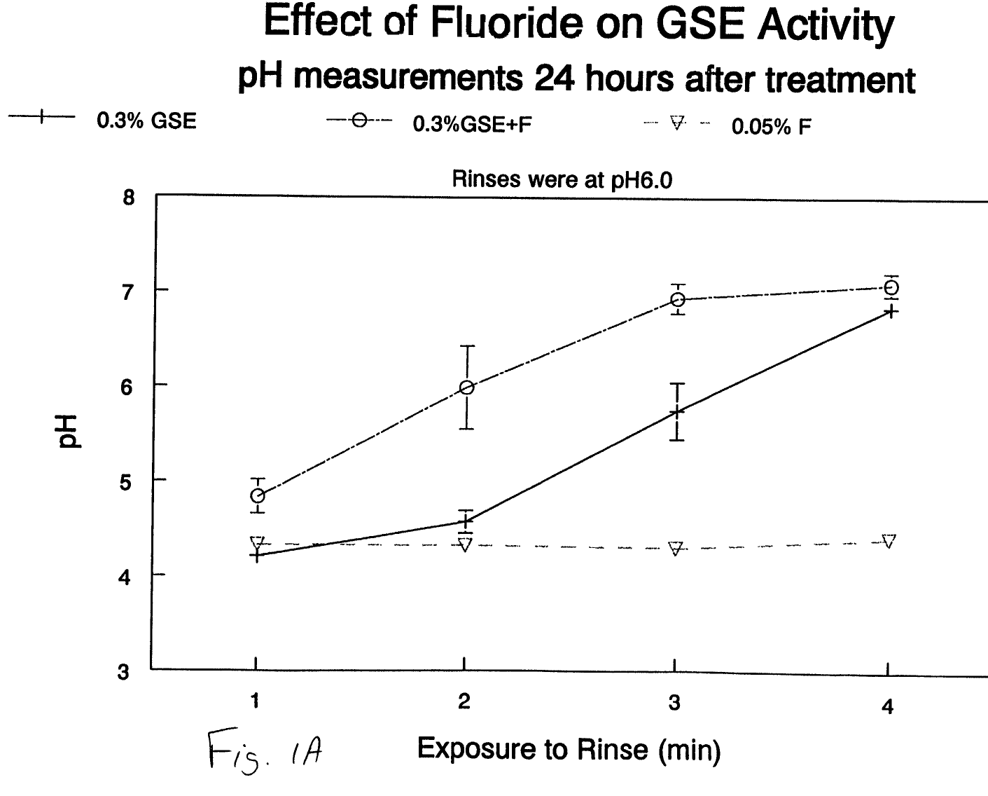

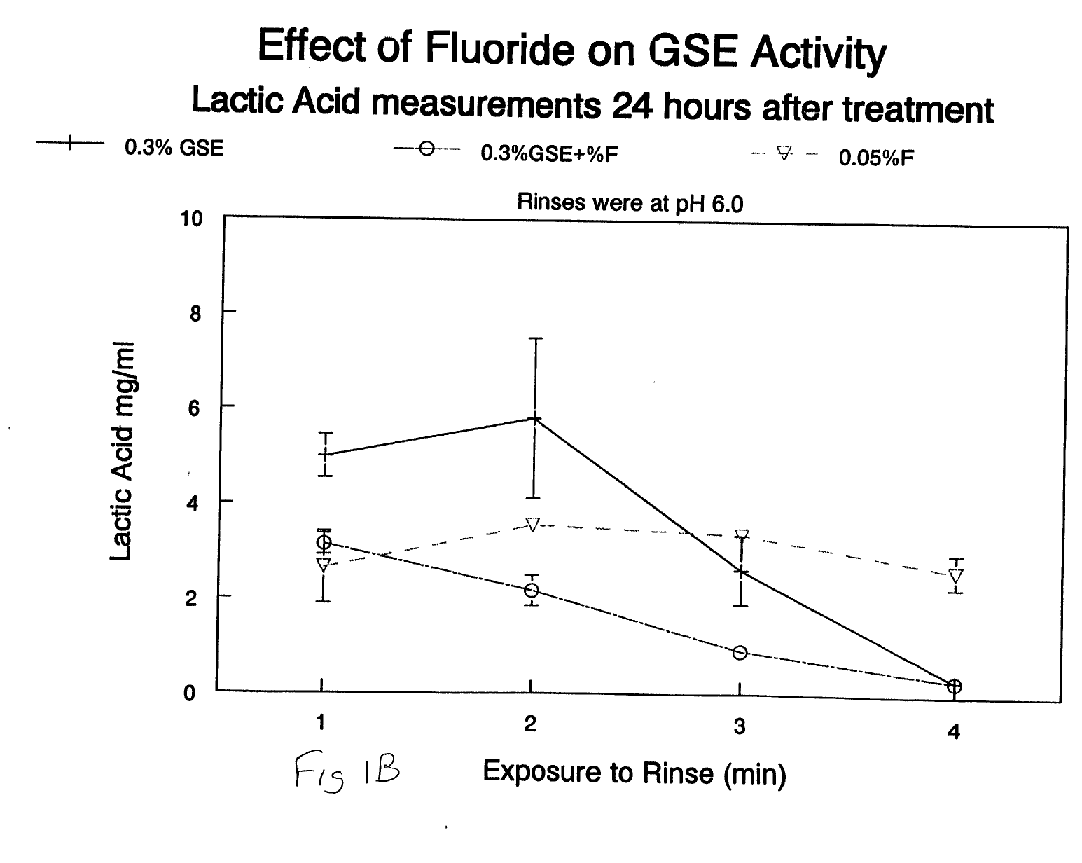

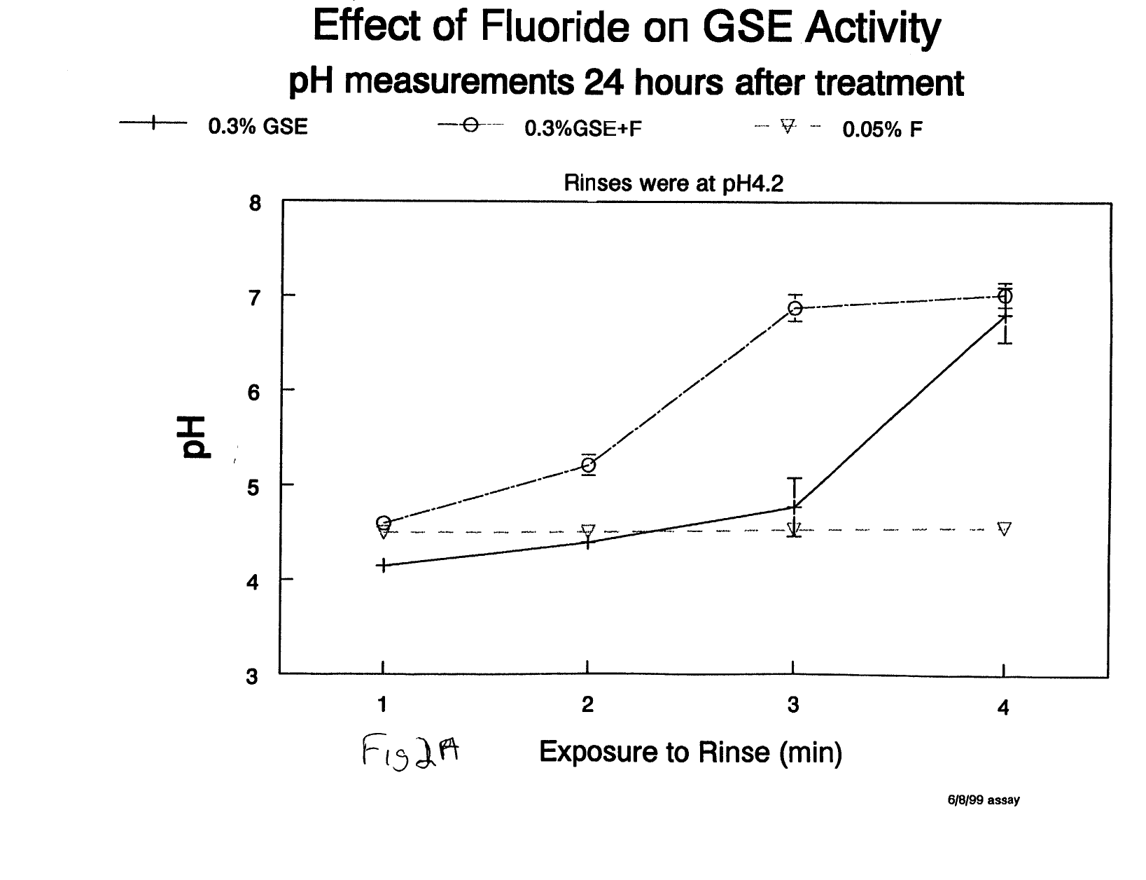

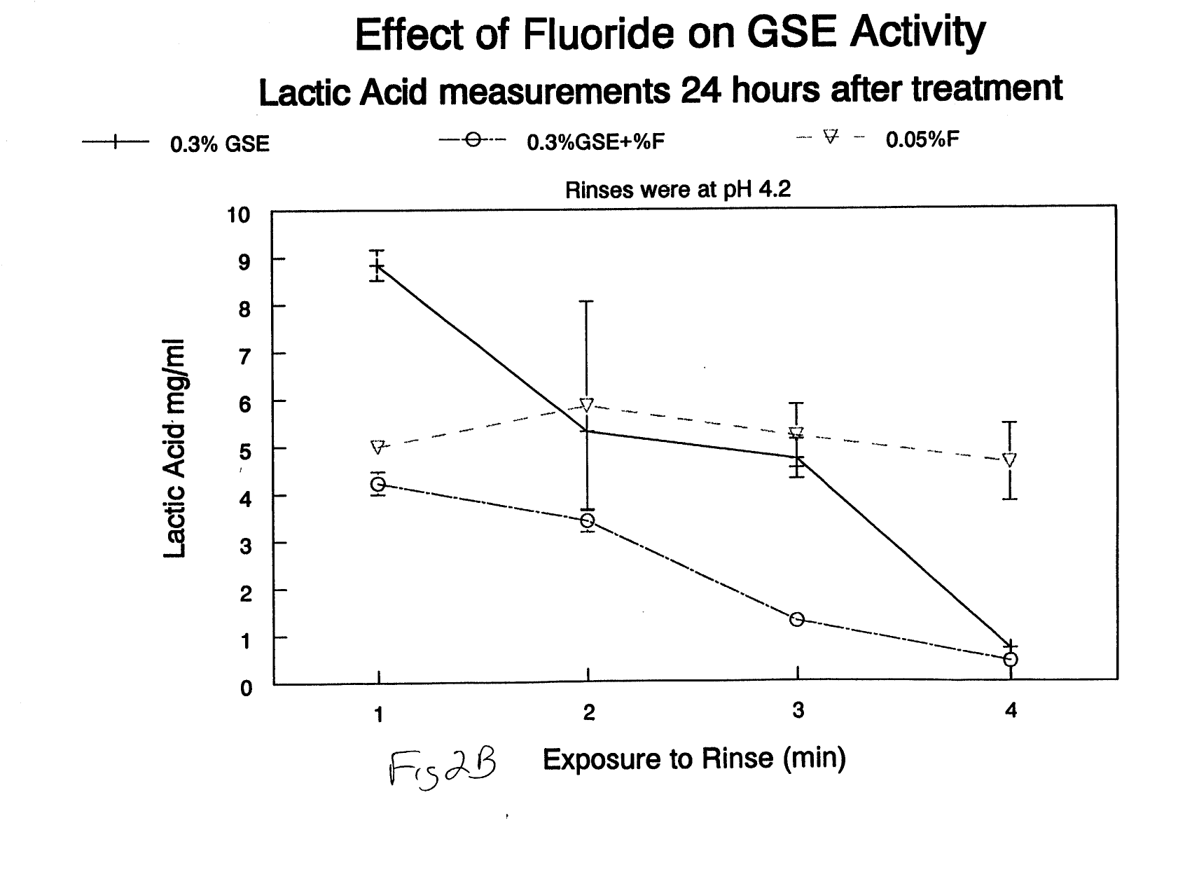

FIGS. 1A and 2A show the pH measurements of wells after exposure to the GSE composition, the GSE/Fluoride composition and the Fluoride composition. In FIG. 1A, the rinses were at pH 6.0 and in FIG. 2A, the rinses were at pH 4.2.

FIGS. 1B and 2B show the lactic acid measurements of wells after exposure to the GSE composition, the GSE/Fluoride composition and the Fluoride composition. In FIG. 1B, the rinses were at pH 6.0 and in FIG. 2B, the rinses were at pH 4.2.

The experiments depicted in the figures demonstrate the enhanced antimicrobial efficacy of compositions comprising GSE and a cation-providing compound (e.g., sodium fluoride) relative to compositions consisting essentially of GSE or the cation-providing compound at two pHs.

While the invention has been described in detail and with reference to specific examples thereof, it will be apparent to one skilled in the art that various changes and modifications can be made therein without departing from the spirit and scope thereof.

What is claimed is:

1. An oral care composition comprising:
a grapefruit seed extract;
an ion-providing compound selected from the group consisting of fluorine-providing compounds and cation-providing compounds;
a flavorant; and
a pharmaceutically acceptable vehicle,
wherein said grapefruit seed extract and said ion-providing compound are synergistically effective to inhibit metabolism of, reduce growth and kill plaque bacteria.

2. The composition of claim 1, wherein said ion-providing compound is a fluorine-providing compound selected from the group consisting of sodium fluoride, potassium fluoride, ammonium fluoride, cuprous fluoride, zinc fluoride, stannic fluoride, stannous fluoride, barium fluoride, sodium fluorosilicate, ammonium fluorosilicate, sodium fluorozirconate, sodium monofluorophosphate, aluminium mono-and difluorophosphate and fluorinated sodium calcium pyrophosphate.

3. The composition of claim 2, wherein said grapefruit seed extract is present in an amount of about 0.001 w/v % to about 10 w/v %, said fluorine-providing compound is present in an amount of about 0.001 wt. % to about 1.0 wt. %, and a ratio of GSE weight to fluorine-providing compounds weight is about 10:1.

4. The composition of claim 1, wherein said ion-providing compound is at least one of a cationic antimicrobial agent and a cationic surfactant.

5. The composition of claim 1, wherein said ion-providing compound is a cation-providing compound selected from the group consisting of cetylpyridinium chloride, domiphen bromide, chlorhexidine, chitosan and ethyl N^{α}-lauryl-L-arginate pyrrolidone-5-carboxylic acid salt.

6. The composition of claim 5, wherein said grapefruit seed extract is present in an amount of about 0.001 w/v % to about 10 w/v %, said cation-providing compound is present in an amount of about 0.001 wt. % to about 1.0 wt. %, and a ratio of GSE weight to cation-providing compounds weight is about 10:1.

7. The composition of claim 1, comprising said fluorine-providing compounds and said cation-providing compounds.

8. The composition of claim 7, wherein said grapefruit seed extract is present in an amount of about 0.001 w/v % to about 10 w/v %, said fluorine-providing compounds and said cation-providing compounds are present in a combined amount of about 0.001 wt. % to about 1.0 wt. %, and a ratio of GSE weight to combined fluorine-providing compounds weight plus cation-providing compounds weight is about 10:1.

9. The composition of claim 1, wherein said vehicle is alcohol-free.

10. The composition of claim 9, wherein said ion-providing compound is a fluorine-providing compound selected from the group consisting of sodium fluoride, potassium fluoride, ammonium fluoride, cuprous fluoride, zinc fluoride, stannic fluoride, stannous fluoride, barium fluoride, sodium fluorosilicate, ammonium fluorosilicate, sodium fluorozirconate, sodium monofluorophosphate, aluminium mono-and difluorophosphate and fluorinated sodium calcium pyrophosphate.

11. The composition of claim 10, wherein said grapefruit seed extract is present in an amount of about 0.001 w/v % to about 10 w/v %, said fluorine-providing compound is present in an amount of about 0.001 wt. % to about 1.0 wt. %, and a ratio of GSE weight to fluorine-providing compounds weight is about 10:1.

12. The composition of claim 9, wherein said ion-providing compound is a cation-providing compound selected from the group consisting of cetylpyridinium chloride, dominiphen bromide, chlorhexidene, chitosan and ethyl N^{α}-lauryl-L-arginate pyrrolidone-5-carboxylic acid salt.

13. The composition of claim 12, wherein said grapefruit seed extract is present in an amount of about 0.001 w/v % to about 10 w/v %, said cation-providing compound is present in an amount of about 0.001 wt. % to about 1.0 wt. %, and a ratio of GSE weight to cation-providing compounds weight is about 10:1.

14. The composition of claim 9, comprising said fluorine-providing compounds and said cation-providing compounds.

15. The composition of claim 14, wherein said grapefruit seed extract is present in an amount of about 0.001 w/v % to about 10 w/v %, said fluorine-providing compounds and said cation-providing compounds are present in a combined amount of about 0.001 wt. % to about 1.0 wt. %, and a ratio of GSE weight to combined fluorine-providing compounds weight plus cation-providing compounds weight is about 10:1.

16. A method for treating an oral cavity to reduce plaque, said method comprising applying to a surface of said oral cavity a composition according to claim 1.

17. The method of claim 16, wherein said ion-providing compound is a fluorine-providing compound selected from the group consisting of sodium fluoride, potassium fluoride, ammonium fluoride, cuprous fluoride, zinc fluoride, stannic fluoride, stannous fluoride, barium fluoride, sodium fluorosilicate, ammonium fluorosilicate, sodium fluorozirconate, sodium monofluorophosphate, aluminium mono-and difluorophosphate and fluorinated sodium calcium pyrophosphate.

18. The method of claim 17, wherein said grapefruit seed extract is present in an amount of about 0.001 w/v % to about 10 w/v %, said fluorine-providing compound is present in an amount of about 0.001 wt. % to about 1.0 wt. %, and a ratio of GSE weight to fluorine-providing compounds weight is about 10:1.

19. The method of claim 16, wherein said ion-providing compound is at least one of a cationic antimicrobial agent and a cationic surfactant.

20. The method of claim 16, wherein said ion-providing compound is a cation-providing compound selected from the group consisting of cetylpyridinium chloride, dominiphen bromide, chlorhexidene, chitosan and ethyl N^{α}-lauryl-L-arginate pyrrolidone-5-carboxylic acid salt.

21. The method of claim 20, wherein said grapefruit seed extract is present in an amount of about 0.001 w/v % to about 10 w/v %, said cation-providing compound is present in an amount of about 0.001 wt. % to about 1.0 wt. %, and a ratio of GSE weight to cation-providing compounds weight is about 10:1.

22. The method of claim 16, wherein said oral care composition comprises said fluorine-providing compounds and said cation-providing compounds.

23. The method of claim 22, wherein said grapefruit seed extract is present in an amount of about 0.001 w/v % to about 10 w/v %, said fluorine-providing compounds and said cation-providing compounds are present in a combined amount of about 0.001 wt. % to about 1.0 wt. %, and a ratio of GSE weight to combined fluorine-providing compounds weight plus cation-providing compounds weight is about 10:1.

24. The method of claim 16, wherein said vehicle is alcohol-free.

25. The method of claim 24, wherein said ion-providing compound is a fluorine-providing compound selected from the group consisting of sodium fluoride, potassium fluoride, ammonium fluoride, cuprous fluoride, zinc fluoride, stannic fluoride, stannous fluoride, barium fluoride, sodium fluorosilicate, ammonium fluorosilicate, sodium fluorozirconate, sodium monofluorophosphate, aluminium mono-and difluorophosphate and fluorinated sodium calcium pyrophosphate.

26. The method of claim 25, wherein said grapefruit seed extract is present in an amount of about 0.001 w/v % to about 10 w/v %, said fluorine-providing compound is present in an amount of about 0.001 wt. % to about 1.0 wt. %, and a ratio of GSE weight to fluorine-providing compounds weight is about 10:1.

27. The method of claim 24, wherein said ion-providing compound is a cation-providing compound selected from the group consisting of cetylpyridinium chloride, domiphen bromide, chlorhexidine, chitosan and ethyl N^{α}-lauryl-L-arginate pyrrolidone-5-carboxylic acid salt.

28. The method of claim 27, wherein said grapefruit seed extract is present in an amount of about 0.001 w/v % to about 10 w/v %, said cation-providing compound is present in an amount of about 0.001 wt. % to about 1.0 wt. %, and a ratio of GSE weight to cation-providing compounds weight is about 10:1.

29. The method of claim 24, wherein said oral care composition comprises said fluorine-providing compounds and said cation-providing compounds.

30. The method of claim 29, wherein said grapefruit seed extract is present in an amount of about 0.001 w/v % to about 10 w/v %, said fluorine-providing compounds and said cation-providing compounds are present in a combined amount of about 0.001 wt. % to about 1.0 wt. %, and a ratio of GSE weight to combined fluorine-providing compounds weight plus cation-providing compounds weight is about 10:1.
